(19) United States
(12) Patent Application Publication
(10) Pub. No.: US 2002/0051799 A1
(43) Pub. Date: May 2, 2002

(54) Glucosylated hydroxystilbene compounds for treating skin conditions

(76) Inventors: Francis Pruche, Senlis (FR); Dominique Bernard, Paris (FR); Bruno Mehul, Villejuif (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/915,353

(22) Filed: Jul. 27, 2001

(30) Foreign Application Priority Data
Jul. 28, 2000 (FR) 00/10008

(51) Int. Cl. A61K 7/00; A61K 31/7034

(52) U.S. Cl. 424/401; 514/25

(57) ABSTRACT

The present invention concerns compositions suitable for topical application, comprising glucosylated hydroxystilbenes. It also concerns a method for slowly releasing hydroxystilbenes into the stratum corneum, by applying a composition comprising glucosylated hydroxystilbenes as a precursor. Finally, it concerns the use of glucosylated hydroxystilbenes to combat signs of cutaneous and hair follicle ageing, to improve the radiance of the skin, to smooth the skin of the face, to treat or prevent wrinkles and fine lines in the skin or to stimulate the epidermal renewal process.

The present invention relates to the use of glucosylated hydroxystilbenes, in particular to glucosylated resveratrol as a hydroxystilbene precursor to compositions for topical application comprising glucosylated hydroxystilbenes, and to a method for slow release of hydroxystilbenes into the stratum corneum, by topical application of a composition comprising glucosylated hydroxystilbenes.

Stilbenes and glucosylated stilbenes are produced in plants, essentially in spermatophytes, and constitute a member of the class of antibiotic molecules known as phytoalexins. A well documented member of this class is resveratrol, or 3,4′,5-trihydroxystilbene.

Resveratrol occurs naturally in many plants and fruits in the simple form (trihydroxystilbene) or in the glucosylated form (piceid, polydatin or 4,5′-dihydroxystilbene-3-O-β-mono-D-glucoside, for example). The two forms, simple and glucosylated, are found in particular in grape skin (Vrhovsek et al., Am. J. Enol. Vitic., vol. 48, n°2, 1997) or in supernatants from in vitro cultures of vitis vinifera (Teguo et al., J. Nat. Prod., 61, 655-657, 1998). Resveratrol is liberated in the presence of β-glucosidases. That reaction occurs naturally in the plant, for example, in grape skin. When fermenting red wine (alcoholic fermentation), this reaction is effected by yeast glycosidases, but it is not complete and a large proportion of glucosylated derivatives remain. The glucosylated form is present in different amounts depending on the wine; certain varieties of Pinot Noir contain exclusively glucosylated hydroxystilbenes (Soleas et al., Clinical Biochemistry, vol. 30, March 1997).

Different in vitro and in vivo studies have demonstrated the advantageous biological properties of hydroxystilbenes, in particular their anti-inflammatory, anti-oxidant and anti-mutagenic properties, and their influence on the metabolism of lipids and on platelet aggregation (Soleas et al., 1997; Jang et al., Science, vol. 275, Jan. 10, 1997).

These properties have been exploited in cosmetic compositions containing those compounds. As an example, cosmetic compositions containing resveratrol and their use in combating the signs of ageing skin, to smooth the skin or to treat wrinkles and fine lines has been described (International application WO-A-99/04747, Unilever, N.V.). A method for obtaining ester derivatives of resveratrol and their use in cosmetic compositions as a resveratrol precursor has also been described (WO-A-99/03816, Caudalie).

In spite of these advantageous properties, hydroxystilbenes possess certain disadvantages in particular as regards their use in the cosmetics field. They may be oxidised by enzymes in the cutaneous flora and thereby lose their advantageous properties.

Further, certain hydroxystilbenes are only soluble and stable in ethanol, which cannot be used in cosmetic formulations. Moreover, hydroxystilbene starting materials are expensive.

In order to exploit the advantageous properties of hydroxystilbenes, principally in the skin, and overcome certain disadvantages of these active principles, the inventors have studied certain derivatives thereof. They have demonstrated that in human, enzymes present in the skin or hair follicles and in particular in the stratum corneum can convert glucosylated hydroxystilbene derivatives to hydroxystilbenes and thus benefit from comparable or even greater advantages compared with those which have been described as a result of topical application of hydroxystilbenes to the skin.

The inventors first experimental approach was directed towards glucosylated resveratrol, which has the advantage of being more stable and soluble than resveratrol and is thus more suitable for use in a cosmetic composition. Further, it is naturally present in certain plants and readily extractable. However, one study has shown that the glucosylated form has an antioxidant activity that is far inferior to the non glucosylated form (Treguo et al., 1998).

The inventors have demonstrated that when used as active principles in cosmetic, dermatological or pharmaceutical compositions that are suitable for topical application, the action of endogenous skin glucosidases, more particularly in the stratum corneum, can liberate active compounds in vivo from hydroxystilbene derivatives, which compounds have advantageous properties and have a favourable effect on microcirculation (better cellular oxygenation), and in particular an in vivo anti-oxidant and/or anti-inflammatory effect.

The invention thus provides a composition for topical application comprising glucosylated hydroxystilbenes with the following general formula I:

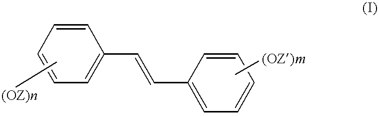

where n is a whole number in the range 1 to 5 inclusive and m is a whole number in the range 0 to 5 inclusive, and Z and Z′, which may be identical or different, represent a hydrogen atom or a glucosyl radical, provided that at least Z or Z′ is a glucosyl radical. These compounds can be in the cis or trans form.

In accordance with the invention, the term “hydroxystilbene” encompasses both compounds with formula I and their hydroxyalkylated derivatives.

Particularly advantageous hydroxystilbenes for producing the compounds of the invention include, for example, glucosylated resveratrol employed under conditions such that it is transformed into resveratrol in vivo.

The expression “glucosylated resveratrol” used in the context of the present patent application encompasses all compounds derived from resveratrol and with formula (II) below:

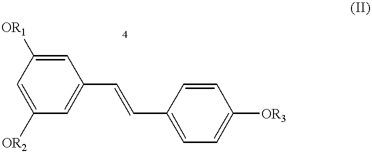

where: R1, R2 and R3, which may be identical or different, represent a hydroxyl group or a glucosyl group, provided that at least R1, R2 or R3 is a glucosyl group.

Examples of glucosylated hydroxystilbenes are:

compounds with the formula given above, in particular 3,4′-dihydroxystilbene-5-O-beta-glucoside; 3,5-dihydroxystilbene-4′-O-beta-glucoside; 4′,5- dihydroxystilbene-3-O-beta-glucoside; 4′-hydroxystilbene-3,5-O-beta-diglucoside; 5-hydroxystilbene-3,4′-O-beta -diglucoside; 3-hydroxystilbene-4′,5-O-beta-diglucoside; and stilbene-3,4′,5-O-beta-triglucoside;

4′-methoxy-3′,5-stilbenediol-3-O-beta-glucoside;

3,5,4′-trihydroxystilbene-2-O-beta-glucoside;

3′,4,5′-trihydroxystilbene-3-O-beta-glucoside;

pinosylvin glucoside, in particular the following compounds: 5-hydroxystilbene-3-O-beta-glucoside; 3-hydroxystilbene-5-O-beta-glucoside; and stilbene-3,5-O-beta-diglucoside.

The invention concerns the D or L glucoside forms of the hydroxystilbenes, or a racemic mixture of these forms. Preferably, the invention concerns the D forms. A highly preferred compound of the invention is 4′,5-dihydroxystilbene-3-O-beta-D-glucoside.

In a particular embodiment of the invention, the compositions comprise a mixture of glucosylated hydroxystilbenes and advantageously a mixture of compounds from the glucosylated resveratrol family.

The invention proposes topical administration of glucosylated hydroxystilbenes, in particular one of the compounds or a mixture of the compounds specifically identified above, for example for a cosmetic or pharmaceutical application.

As a result, the invention provides a process for slowly releasing hydroxystilbenes into the stratum corneum by topical application of a composition comprising glucosylated hydroxystilbenes, in particular one of the compounds or a mixture of the compounds specifically identified above.

To carry out the invention, the glucosylated hydroxystilbenes, in particular one of the compounds or a mixture of the compounds specifically identified above, can be extracted from plants or parts of plants. They can also be synthesised chemically.

When the glucosylated hydroxystilbenes are isolated from plants or plant material containing them in order to prepare the compositions of the invention, plants from the following families can be used: Vitaceae, Ombellifereae, Myrtaceae, Dipterocarpaceae, Cyperaceae, Gneticae, Legumes, Gramineae, Sericeae, Haemodoraceae, Musaceae, Polygonaceae, Pinaceae, Crupressaceae, Cesalpiniaceae, Poaceae and Solanaceae. More particularly, they are isolated from vitus vinifera or from polygonum cuspidatum tissue, preferably from grape skin. They can also be extracted from products derived from grapes, such as wine.

Glucosylated hydroxystilbenes, in particular one of the compounds or a mixture of the compounds specifically identified above, can be extracted and purified by means of the extraction procedure described by A. L. Waterhouse (Petrochemistry, vol. 37, p. 571 (1994)).

A preferred form of hydroxystilbenes for producing the compositions of the invention is glucosylated resveratrol. A simple method for obtaining a fraction enriched in glucosylated resveratrol consists in extraction with methanol and/or ethanol/water. As an example, 100 g of polygonum cuspidatum is mixed with 800 ml of water and 200 ml of ethanol, the mixture is stirred vigorously for 12 hours at 4° C. then filtered. This filtrate can be delipided with petroleum ether, for example, and may or may not be taken up in water then evaporated off.

Glucosylated resveratrol can also be extracted from in vitro cultures of vitis vinifera cells. Methods for extracting glucosylated resveratrol have been described by Teguo et al., (Teguo et al., 1998) or in International patent application WO-A-99/03816.

In accordance with the invention, the glucosylated hydroxystilbenes, in particular one of the compounds or a mixture of the compounds specifically identified above, represents in the range 0.01% to 10% of the total composition weight, preferably in the range 0.1% to 5% of the total composition weight.

The compositions of the invention are in the form of a cream, ointment, emulsion, gel or lotion.

One advantage of the compositions of the invention is the possibility of modulating the kinetics of the enzymatic reaction resulting in liberation of hydroxystilbenes from glucosylated hydroxystilbenes (bioconversion), in particular from one of the compounds or a mixture of the compounds specifically identified above, and more particularly from glucosylated resveratrol.

The compositions may comprise glucosidase activators or inhibitors or any product resulting in a modulation of the bioconversion kinetics. Thus, activators are added to the cosmetic or pharmaceutical composition to stimulate the activity of endogenous glucosidases. An example of such an activator is 1-O-methyl-β-D-glucopyranoside. These activators represent in the range 0.01% to 10% of the total composition weight, preferably in the range 0.1% to 5% of the total composition weight.

In a preferred implementation of the invention, the pH of the composition of the invention is close to that of skin, preferably from 4 to 7. This results in good compatibility and tolerance of the composition of the invention with skin.

The composition of the invention is intended for topical application and, suitably, comprises a physiologically acceptable medium. The term “physiologically acceptable medium” means a medium that is compatible with skin, mucosa (including the inside of the eyelids and the lips), the nails and/or keratinous fibers (hair and eyelashes).

Further, in known manner, the compositions of the invention may contain additives that are usual in the cosmetic and/or pharmaceutical field, such as hydrophilic or lipophilic agents, preservatives, anti-oxidants, fragrances, fillers, coloring substances (pigments or colorants), sun screens, solvents or lipidic vesicles. These additives are used in proportions that are normal in the cosmetic or dermatological field, for example 0.01% to 20% of the total composition weight and, depending on their nature, they are introduced into an aqueous phase or into an oily phase of the composition, or into vesicles.

Clearly, the skilled person will be careful to select any additive or additives so that the advantageous properties of the composition of the invention are not altered or are not substantially altered by the envisaged additives.

The sun screen is preferably selected from organic filters and/or mineral filters.

Examples of organic filters that can be cited are cinnamic derivatives, salicylic derivatives, camphor derivatives, triazine derivatives, benzophenone derivatives, dibenzoylmethane derivatives, β, β-diphenylacrylate derivatives, p-aminobenzoic acid derivatives, polymer filters and silicone filters described in International patent application WO-A-93/04665, and organic filters described in European patent application EP-A-0 487 404.

Examples of mineral filters that can be cited are pigments or nanopigments (average size of 20 primary particles: generally in the range 5 nm to 10 nm, preferably in the range 10 nm to 50 nm) of coated or uncoated metal oxides, such as titanium oxide nanopigments (amorphous or crystalline, in the form of rutile and/or anatase), iron, zinc, zirconium or cerium which are all photoprotective agents that are well known per se, acting by physically blocking UV radiation (reflection and/or diffusion). Alumina and/or aluminium stearate constitute conventional coating agents. Such coated or uncoated metal oxide nanopigments have been described in particular in patent applications EP-A-0 518 772 and EP-A-0 518 773.

Examples of complementary sun screens that are active in the UV-A and/or UV-B regions that can be cited are:

p-aminobenzoic acid;

oxyethylenated p-aminobenzoate (25 mol);

2-ethylhexyl p-dimethylaminobenzoate;

N-oxypropylenated ethyl p-aminobenzoate;

glycerol p-aminobenzoate;

homomenthyl salicylate;

2-ethylhexyl salicylate;

triethanolamine salicylate;

4-isopropylbenzyl salicylate;

4-ter-butyl-4′-methoxy-dibenzoylmethane (PARSOL 1789 from GIVAUDAN ROURE);

2-ethylhexyl p-methoxycinnamate (PARSOL MCX from GIVAUDAN ROURE);

4-isopropyl-dibenzoylmethane (EUSOLEX 8020 from MERCK);

menthyl anthranilate;

2-ethylhexyl-2-cyano-3,3′-diphenylacrylate (UVINUL N539 from BASF);

ethyl-2-cyano-3,3′-diphenylacrylate;

2-phenylbenzimidazole 5-sulphonic acid and salts thereof;

3-(4′-trimethylammonium)-benzylidene-bornan-2-on-methylsulphate;

2-hydroxy-4-methoxybenzophenone (UVINUL MS 40 from BASF);

2-hydroxy-4-methoxybenzophenone-5-sulphonate (UVINUL MS40 from BASF);

2,4-dihydroxybenzophenone (UVINUL 400 from BASF); sold by SEPPIC under the trade name SEPIGEL 305, and acrylamidomethylpropanesulphonic acid polymers that are at least partially cross linked, such as the product sold by HOECHST with the trade name HOSTACERIN AMPS. These gelling agents are generally used in concentrations of 0.1% to 10%, preferably 0.1% to 5%, more preferably 0.1% to 3% of the total composition weight.

The invention also concerns a cosmetic treatment method for controlling skin pigmentation, to combat signs of cutaneous ageing and of the hair follicle, to improve the radiance of the skin, to smooth the skin of the face, to treat or prevent wrinkles and fine lines in the skin or to stimulate the epidermal renewal process, consisting of applying a composition as defined above to the skin.

Finally, it concerns the use of glucosylated hydroxystilbenes, in particular one of the compounds or a mixture of the compounds specifically identified above as a hydroxystilbene precursor by topical application of cosmetic or pharmaceutical compositions containing it.

The following examples illustrate the liberation of resveratrol from glucosylated resveratrol in the presence of soluble extracts of the stratum corneum and also illustrate a mode of producing the composition so the invention. They are not limiting in nature.

### EXAMPLE 1

This example demonstrates that it is possible to liberate resveratrol from glucosylated resveratrol in the presence of an extract of stratum corneum or a hair follicle homogenate. It also demonstrates that adding a glucosidase activator can significantly improve the hydrolysis reaction.

A glucosylated resveratrol (4′,5-dihydroxystilbene-3-O-beta-D-glucoside) (APIN Chemicals N17555p, 29 D Milton Park, Abingdon, Oxon, United Kingdom) was incubated in the presence of an extract of stratum corneum or of a homogenate of hair follicles that had been removed by plucking. Under precise experimental conditions, the affinity of the resveratrol for tyrosinase was higher than for glucosylated resveratrol. This property was advantageously employed to measure the resveratrol liberated in the presence of tyrinosase, determined at 255 nm. This method was adapted from the method described by A. A. Calderon: “A Spectrophotometric Assay for Quantitative Analysis of the Oxidation of 4-hydroxystilbene by Peroxidase-H₂O₂ Systems: J. of Biochem. and Biophys. (1990) Methods 20: 171-180.

1. Apparatus and solutions used

4′,5-dihydroxystilbene-3-O-beta-D-glucoside:1 mM in ethanol;

resveratrol (SIGMA R501 0): 1 mM in ethanol;

tyrosinase (SIGMA T7755) 600 units/ml PBS;

activator: 1-0-methyl-β-D-glucopyranoside (Biosynth: Biochemica et synthetica):1 mM in PBS;

PBS buffer, pH 7.2.

An extract of stratum corneum cells was removed by scraping in the presence of PBS (10 ml over a zone of about 20 cm²), then filtered through a 0.22 μm filter to obtain a stratum corneum solution.

5 human follicles were removed by plucking and homogenised in 100 μl of PBS to obtain a hair follicle homogenate.

400 μl of stratum corneum solution was incubated in the presence of resveratrol glucoside (50 μl) in the presence and absence of activator (25 μl).

20 μl of hair follicle homogenate was incubated in the presence of resveratrol glucoside (50 μl) in the presence and absence of activator (10 μl).

The volume was adjusted to 500 μl with PBS and incubation was carried out for 5 hours at 37° C. to 25° C.

A 200 μl aliquot was removed and 10 μl of tyronisase was added.

Two independent measurements were carried out by determining the optical density at 255 nm over 10 minutes and calculating the slope of the graph (60 to 300 seconds).

Under the same conditions, a calibration curve was produced using resveratrol (1; 2.5; 5; 7.5 and 10 μl) in a volume adjusted to 200 ml.

2. Results

Table 1 below gives the results of n vitro tests carried out with glucosylated resveratrol alone or, by way of example, in the presence of glucosidase activator (1-O-methyl-β-D-glucopyranoside) at two different concentrations.

TABLE 1
STRATUM CORNEUM

| | |
|---|---|
| Resveratrol liberated: | |
| no activator | 2.85 nmoles |
| 0.5 mM activator | 5.65 nmoles (+98%) |
| 0.1 mM activator | 4.00 nmoles (+40%) |

HAIR FOLLICLE HOMOGENATE

| | |
|---|---|
| Resveratrol liberated: | |
| no activator | 6.15 nmoles |
| 0.5 mM activator | 7.15 nmoles (+16%) |
| 0.1 mM activator | 6.70 nmoles (+9%) |

The results obtained with in vitro tests demonstrate significant liberation of resveratrol from glucosylated resveratrol. This liberation could also be increased in the presence of glucosidase activators. The in vitro tests also demonstrated a resveratrol action at relatively low concentrations of less than 10 μm. Limited but continuous liberation over time of resveratrol from glucosylated resveratrol is thus particularly suitable for cosmetic applications.

The percentages shown in brackets indicate the percentage increase in the number of molecules of resveratrol liberated compared with the control in the absence of activators.

### EXAMPLE 2

### Formulation for a Cosmetic Composition in Accordance with the Invention

A cosmetic composition in accordance with the invention was formulated as follows:

Treatment cream

| | |
|---|---|
| Cetyl alcohol | 1.05% |
| PEG 20 stearate (Myrj 49 sold by ICI) | 2% |
| Cyclomethicone | 6% |
| 4′,5-dihydroxystilbene-3-O-beta-mono-D-glucoside | 0.5% |
| 1-O-methyl-beta-D-glucopyranoside | 0.3% |
| Carbomer | 0.6% |
| Glycerine | 3% |
| Triethanolamine | 1% |
| Preservatives | 0.5% |
| Demineralised water, qsp | 100% |

1. A composition for topical application, comprising glucosylated hydroxystilbenes with the general formula: 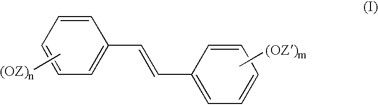
where n is a whole number in the range 1 to 5 inclusive and m is a whole number in the range 0 to 5 inclusive, and Z and Z′, which may be identical or different, represent a hydrogen atom or a glucosyl radical, provided that at least Z or Z′ is a glucosyl radical.

2. A composition according to claim 1, in which the glucosylated hydroxystilbene is a “glucosylated resveratrol” with general formula II: 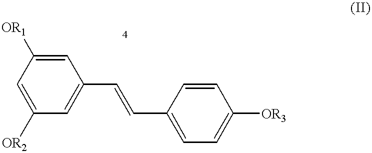
where: R1, R2 and R3, which may be identical or different, represent a hydroxyl group or a glucosyl group, provided that at least R1, R2 or R3 is a glucosyl group.

3. A composition according to claim 1, in which the glucosylated hydroxystilbene or mixture of compounds is selected from the group formed by the following compounds:
3,4′-dihydroxystilbene-5-O-beta-glucoside;
3,5-dihydroxystilbene-4′-O-beta-glucoside;
4′,5-dihydroxystilbene-3-O-beta-glucoside;
4′-hydroxystilbene-3,5-O-beta-diglucoside;
5- hydroxystilbene-3,4′-O-beta-diglucoside;
3- hydroxystilbene-4′,5-O-beta-diglucoside;
stilbene-3,4′,5-O-beta-triglucoside;
4′-methoxy-3′,5-stilbenediol-3-O-beta-glucoside;
3,5,4′-trihydroxystilbene-2-O-beta-glucoside;
3′,4,5′-trihydroxystilbene-3-O-beta-glucoside;
5-hydroxystilbene-3-O-beta-glucoside;
3-hydroxystilbene-5-O-beta-glucoside;
stilbene-3,5-O-beta-diglucoside.

4. A composition according to claim 2, in which the glucosylated resveratrol is 4′,5-dihydroxystilbene-3-O-beta-D-glucoside.

5. A composition according to claim 2 or claim 4, characterized in that the glucosylated resveratrol is isolated from cells of vitis vinifera cultivated in vitro.

6. A composition according to any one of claims 1 to 5, characterized in that it also comprises a glucosidase activator.

7. A composition according to claim 6, characterized in that the glucosidase activator is 1-O-methyl-β-D-glucopyranoside.

8. A composition according to claim 6 or claim 7, characterized in that it comprises in the range 0.01% to 10% of glucosidase activator, preferably in the range 0.1% to 5%.

9. A composition according to any one of claims 1 to 5, characterized in that it also comprises a glucosidase inhibitor.

10. A composition according to any one of claims 1 to 9, characterized in that the glucosylated hydroxystilbene is extracted from plants.

11. A composition according to claim 10, characterized in that the glucosylated resveratrol is extracted from vitis vinifera tissue or from polygonum cuspidatum tissue, in particular from grape skin.

12. A composition according to any one of claims 1 to 9, characterized in that the glucosylated hydroxystilbene is extracted from wine.

13. A composition according to any one of claims 1 to 12, characterized in that it is formulated at a pH in the range 4 to 7.

14. A composition according to any one of claims 1 to 13, characterized in that it comprises in the range 0.01% to 10% of glucosylated hydroxystilbenes, preferably in the range 0.1% to 5%.

15. A cosmetic method for liberating hydroxystilbenes from stratum corneum by applying a composition as defined in any one of claims 1 to 14 to the skin.

16. Use of glucosylated hydroxystilbenes as a hydroxystilbene precursor to control skin pigmentation, to combat signs of cutaneous ageing and hair follicle ageing, to improve the radiance of the skin, to smooth the skin of the face, to treat or prevent wrinkles and fine lines in the skin, or to stimulate the epidermal renewal process, by applying a composition suitable for topical application according to any one of claims 1 to 14 to the skin.

17. Use of glucosylated hydroxystilbenes as a hydroxystilbene precursor in a method according to claim 15.

18. Use of glucosylated hydroxystilbenes as a hydroxystilbene precursor in a composition for topical application.
